(19) United States
(12) Patent Application Publication
(10) Pub. No.: US 2026/0354699 A1
(43) Pub. Date: Nov. 12, 2026

(54) OPTICAL INSPECTION DEVICE AND OPTICAL FIBER STRUCTURE

(71) Applicant: FURUKAWA ELECTRIC CO., LTD., Tokyo (JP)

(72) Inventors: Kumi KAWASHIMA, Tokyo (JP); Junichi HASEGAWA, Tokyo (JP); Yoshiki NOMURA, Tokyo (JP); Kyosuke YAMAUCHI, Tokyo (JP); Saori WATANABE, Tokyo (JP)

(73) Assignee: FURUKAWA ELECTRIC CO., LTD., Tokyo (JP)

(21) Appl. No.: 19/515,112

(22) PCT Filed: Aug. 29, 2024
(86) PCT No.: PCT/JP2024/030924

§ 371 Date: Feb. 27, 2026

(30) Foreign Application Priority Data
Aug. 29, 2023 (JP) 2023-138962

(51) Int. Cl. A61B 5/026; A61B 5/00; A61B 17/12

(52) U.S. Cl. CPC A61B 5/0261; A61B 5/6852; A61B 17/1204; A61B 17/12109; A61B 17/12136; A61B 2505/05; A61B 2562/0233

(57) ABSTRACT

An optical inspection device includes a light source, a light emission optical fiber having a first light guide portion through which light emitted from the light source propagates and a first light emitting portion which emits the emitted light onto a detection target. A light reception optical fiber has a first light receiving portion for receiving reflected light resulting from the emitted light emitted from the first light emitting portion being reflected by the detection target and a second light guide portion through which the reflected light received by the first light receiving portion propagates, and a light detecting portion for detecting the reflected light that has propagated through the second light guide portion. An optical fiber structure is formed by arranging the first light emitting portion and the first light receiving portion so as to be mutually offset in the fiber axial direction.

### TECHNICAL FIELD

The present invention relates to an optical examination device and an optical fiber structure.

### BACKGROUND ART

An optical examination device which irradiates a detection object with light and detects reflected light is configured to: propagate light emitted from a light source to a light emitting portion using an optical fiber; irradiate the detection object with the light from the light emitting portion; propagate the reflected light from the detection object received by a light receiving portion to a light detecting portion using an optical fiber; process the light detected by the light detecting portion; and examine the detection object, and is used in a variety of fields including a medical field.

For example, Patent Document 1 discloses a laser blood flowmeter including a blood flowmeter body, an irradiating optical fiber and a light-receiving optical fiber connected to the blood flowmeter body, and a probe to which tips of the irradiating optical fiber and the light-receiving optical fiber are fixed in parallel, the laser blood flowmeter being configured to irradiate a biological tissue with laser light from the blood flowmeter body through the irradiating optical fiber, and to transmit scattered light within the biological tissue as blood flow information to the blood flowmeter body through the light-receiving optical fiber. The probe for an optical fiber used in the laser blood flowmeter includes a reflective fixing plate provided with a fixing means for fixing to the biological tissue, the probe being formed by fixing the irradiating optical fiber and the light-receiving optical fiber in parallel to a reflective surface of the reflective fixing plate and being configured so that reflected laser light output from the irradiating optical fiber and the scattered light within the biological tissue are reflected by the reflective surface of the reflective fixing plate.

In addition, Patent Document 2 discloses a medical system including: a catheter including a distal-end assembly for performing medical surgery on a tissue in a cavity of an organ of a patient, the distal-end assembly including an optical fiber configured to guide irradiation light (transmitted light) to interact with the tissue in the cavity, and to guide reflected light (return light) that interacts with the tissue; a light source configured to produce the irradiation light; a detector configured to measure the reflected light; a circulator configured to couple the irradiation light from the light source to the optical fiber and to couple the reflected light from the optical fiber to the detector; and a processor configured to identify a contact of the distal-end assembly with the tissue based on the reflected light measured by the detector and to indicate the identified contact to a user.

### CITATION LIST

### Patent Documents

Patent Document 1: Japanese Unexamined Patent Application, Publication No. H10-118039

Patent Document 2: Japanese Unexamined Patent Application, Publication No. 2022-126610

### DISCLOSURE OF THE INVENTION

### Problems to be Solved by the Invention

When temporarily blocking blood flow in a blood vessel during surgery, a surgery method is used to block the blood flow in such a manner that a balloon catheter is inserted into the blood vessel and the balloon is inflated. In fields other than the medicine, such as water-supply works, when temporarily stopping the flow of water in a pipe, a construction method is used to stop the water in such a manner that an airbag is inserted into the pipe and the airbag is inflated (airbag stopper method). In these surgery method and construction method, it is considered that a light-emitting optical fiber and a light-receiving optical fiber are arranged inside a balloon catheter or an airbag, and blocking of the blood vessel or the pipe is detected using a difference in reflectance of light between a blood vessel wall serving as a detection object and blood or a difference in reflectance of light due to the presence or absence of water between a pipe wall serving as a detection object and the airbag.

However, the laser blood flowmeter disclosed in Patent Document 1 measures blood flow by being fixed to a surface to be measured of the biological tissue, such as a body surface, using the reflective fixing plate, and is not intended to be inserted into a thin tube such as a blood vessel.

The medical system disclosed in Patent Document 2 is configured by one optical fiber, and uses the circulator to separate the irradiation light, which is emitted from the light source, from the reflected light from the detection object. When the irradiation light and the reflected light are propagated through only one optical fiber, as in the medical system, reflected light (hereinafter, referred to as Fresnel reflected light) occurs due to Fresnel reflection at a tip of the optical fiber, and the Fresnel reflected light becomes a large noise compared to the reflected light reflected from the detection object, resulting in a problem of a lower SN ratio (signal-to-noise ratio).

An object of the present invention is to provide an optical examination device and an optical fiber structure that can be inserted into a tubular body, for example, when an examination object is a tubular body, particularly a thin tubular body such as a blood vessel, and that can prevent noise due to Fresnel reflected light and improve an SN ratio when receiving reflected light reflected from the examination object.

### Means for Solving the Problems

The present inventors have found that when a light-emitting optical fiber for propagating light emitted from a light source and a light-receiving optical fiber for propagating reflected light received from a detection object (inspection object) are used; a first light emitting portion of the light-emitting optical fiber and a first light receiving portion of the light-receiving optical fiber are arranged to be offset from each other in a fiber axial-direction X; and a tip of the first light receiving portion is preferably located closer to a first light guide portion than a tip of the first light emitting portion, or the tip of the first light emitting portion is preferably located closer to a second light guide portion than a tip of the first light receiving portion, and the first light receiving portion of the light-receiving optical fiber can effectively receive the reflected light without directly receiving irradiation light emitted from the first light emitting portion of the light-emitting optical fiber, thereby making it possible to improve an SN ratio when receiving the reflected light, and thus have completed the present invention.

In order to achieve the above object, the present invention has main configurations as follows.
(1) An optical inspection device including: a light source; at least one light-emitting optical fiber including a first light guide portion that propagates light emitted from the light source and a first light emitting portion that irradiates a detection object with the light propagated through the first light guide portion, as irradiation light; at least one light-receiving optical fiber including a first light receiving portion that receives reflected light obtained when the irradiation light emitted from the first light emitting portion is reflected by the detection object and a second light guide portion that propagates the reflected light received by the first light receiving portion; and a light detecting portion that detects the reflected light propagated through the second light guide portion, in which, the light-emitting optical fiber and the light-receiving optical fiber form an optical fiber structure in which the first light emitting portion and the first light receiving portion are arranged to be offset from each other in a fiber axial-direction.
(2) The optical inspection device according to (1) above, in which the irradiation light is emitted from a side surface of the first light emitting portion.
(3) The optical inspection device according to (1) or (2) above, in which the first light receiving portion of the light-receiving optical fiber has a longer length than the first light emitting portion of the light-emitting optical fiber.
(4) The optical inspection device according to any one of (1) to (3) above, in which the light-emitting optical fiber and the light-receiving optical fiber form the optical fiber structure in which the first light emitting portion and the first light receiving portion are arranged to be partially offset from each other in the fiber axial-direction.
(5) The optical inspection device according to any one of (1) to (4) above, in which the light-emitting optical fiber and the light-receiving optical fiber are respectively arranged so that a tip of the first light emitting portion and a tip of the first light receiving portion are at the same position in the fiber axial-direction.
(6) The optical inspection device according to any one of (1) to (5) above, in which the light-receiving optical fiber includes a plurality of the first light receiving portions.
(7) The optical inspection device according to any one of (1) to (6) above, in which the light-emitting optical fiber includes a plurality of the first light emitting portions.
(8) The optical inspection device according to any one of (1) to (7) above, in which one or both of the first light emitting portion and the first light receiving portion are provided in an intermediate part of the light-emitting optical fiber or the light-receiving optical fiber.
(9) The optical inspection device according to any one of (1) to (8) above, in which the light-emitting optical fiber and the light-receiving optical fiber are made of a quartz fiber or a plastic fiber.
(10) An optical fiber structure including: at least one light-emitting optical fiber including a first light guide portion that propagates light emitted from a light source and a first light emitting portion that irradiates a detection object with the light propagated through the first light guide portion, as irradiation light; and at least one light-receiving optical fiber including a first light receiving portion that receives reflected light obtained when the irradiation light emitted from the first light emitting portion is reflected by the detection object and a second light guide portion that propagates the reflected light received by the first light receiving portion, in which, the light-emitting optical fiber and the light-receiving optical fiber form an optical fiber structure in which the first light emitting portion and the first light receiving portion are arranged to be offset from each other in a fiber axial-direction.

### Effects of the Invention

According to the present invention, it is possible to provide an optical examination device and an optical fiber structure that can be inserted into a tubular body, for example, when an examination object is a tubular body, particularly a thin tubular body such as a blood vessel, and that can prevent noise due to Fresnel reflected light and improve an SN ratio when receiving reflected light reflected from the examination object.

### BRIEF DESCRIPTION OF THE DRAWINGS

FIG. 1 is a plan view schematically showing a configuration of an optical examination device and an optical fiber structure according to a first embodiment;

FIGS. 2A and 2B are views showing a positional relationship between the optical fiber structure and a tube when the optical fiber structure of the optical examination device according to the first embodiment is inserted into the tube, which is a detection object, where FIG. 2A is an enlarged longitudinal cross-sectional view of a main portion of the optical fiber structure, and FIG. 2B is a cross-sectional view taken along line I-I′ in FIG. 2A, showing optical paths of irradiation light emitted from a first light emitting portion and reflected light from the tube received by a first light receiving portion;

FIG. 3 is a plan view showing a structure of an optical fiber structure according to a second embodiment;

FIG. 4 is a cross-sectional view showing an arrangement of a light-emitting optical fiber and a light-receiving optical fiber that form an optical inspection device and an optical fiber structure according to a third embodiment;

FIGS. 5A and 5B are plan views showing a structure of an optical fiber structure according to a fourth embodiment, where FIG. 5A shows a case where a tip of a first light receiving portion is arranged to be located closer to a first light guide portion than a tip of a first light emitting portion, and FIG. 5B shows a case where the tip of the first light emitting portion is arranged to be located closer to a second light guide portion than the tip of the first light receiving portion;

FIGS. 6A and 6B are plan views showing a structure of an optical fiber structure according to a fifth embodiment, where FIG. 6A shows a case where a length dimension of a first light receiving portion of a light-receiving optical fiber is longer than a length dimension of a first light emitting portion of a light-emitting optical fiber, and FIG. 6B shows a case where a length dimension of a first light emitting portion of a light-emitting optical fiber is longer than a length dimension of a first light receiving portion of a light-receiving optical fiber;

FIGS. 7A and 7B are plan views showing a structure of an optical fiber structure according to a sixth embodiment, where FIG. 7A shows a case where a light-receiving optical fiber includes a plurality of first light receiving portions, and FIG. 7B shows a case where a light-emitting optical fiber includes a plurality of first light emitting portions;

FIG. 8 is a plan view showing a structure of an optical fiber structure according to a seventh embodiment;

FIGS. 9A to 9D are longitudinal cross-sectional views of a first light emitting portion of a light-emitting optical fiber showing various aspects, where FIG. 9A shows a case where a rough surface is formed on a surface of a cladding of the light-emitting optical fiber to form a first light emitting portion, FIG. 9B shows a case where a cladding of a light-emitting optical fiber is completely removed by scraping in a radial direction and a light-diffusion resin layer is formed on a surface of the cladding removed by scraping to form a first light emitting portion, FIG. 9C shows a case where a cladding of a light-emitting optical fiber is partially removed by scraping in a radial direction and a light-diffusion resin layer is formed on a surface of the cladding removed by scraping to form a first light emitting portion, and FIG. 9D shows a case where a coreless fiber or a light diffusion fiber is fusion-spliced to a tip of a light-emitting optical fiber;

FIGS. 10A and 10B are longitudinal cross-sectional views of a first light emitting portion of a light-emitting optical fiber showing various aspects, where FIG. 10A shows a case where tips of a core and a cladding of a light-emitting optical fiber is cut into a conical shape to form a first light emitting portion, and FIG. 10B shows a case where a core and a cladding of a light-emitting optical fiber are cut obliquely to form a first light emitting portion;

FIGS. 11A and 11B are longitudinal cross-sectional views of a first light receiving portion of a light-receiving optical fiber showing various aspects, where FIG. 11A shows a case where a rough surface is formed on a surface of a cladding of a light-receiving optical fiber to form a first light receiving portion, and FIG. 11B shows a case where a core of a light-receiving optical fiber is left and a cladding is removed by scraping to form a first light receiving portion; and

FIGS. 12A to 12D are longitudinal cross-sectional views of a first light receiving portion of a light-receiving optical fiber showing various aspects, where FIG. 12A shows a case where a rough surface is formed on a surface of a cladding of a light-receiving optical fiber to form a first light receiving portion, FIG. 12B shows a case where a cladding of a light-receiving optical fiber is completely removed by scraping in a radial direction and a light-diffusion resin layer is formed on a surface of the cladding removed by scraping to form a first light receiving portion, FIG. 12C shows a case where a cladding of a light-receiving optical fiber is partially removed by scraping in a radial direction and a light-diffusion resin layer is formed on a surface of the cladding removed by scraping to form a first light receiving portion, and FIG. 12D shows a case where a coreless fiber or a light diffusion fiber is fusion-spliced to a tip of a light-receiving optical fiber.

### PREFERRED MODE FOR CARRYING OUT THE INVENTION

Next, optical examination devices and optical fiber structures according to some embodiments of the present invention will be described.

### First Embodiment

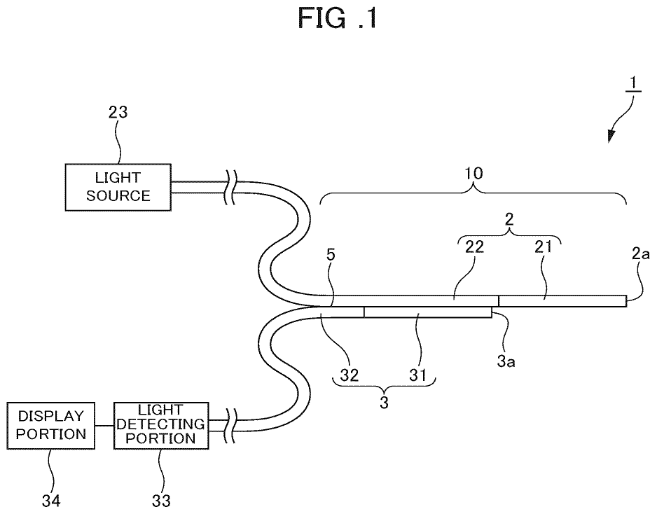

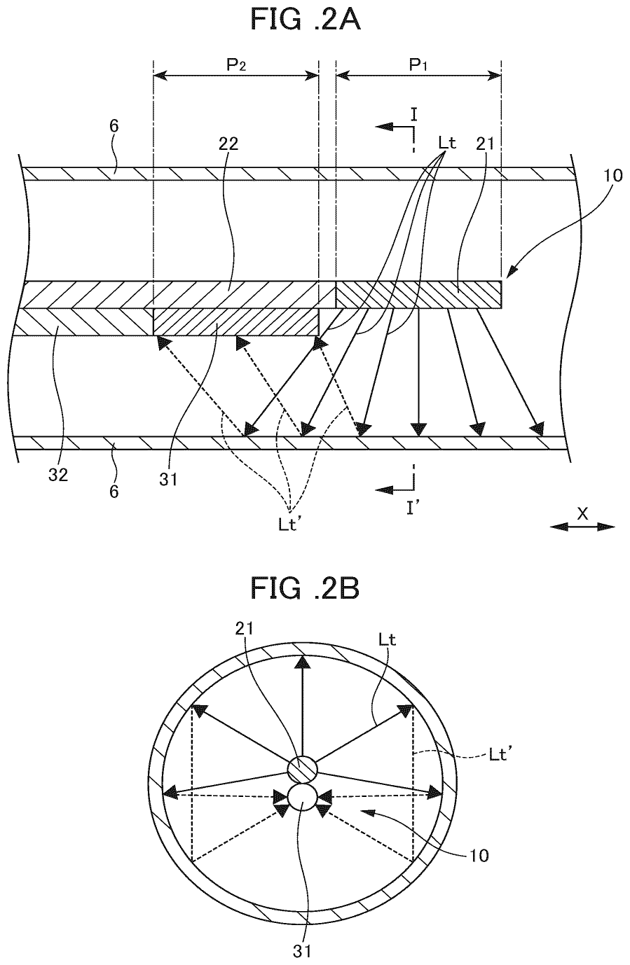

FIG. 1 is a plan view schematically showing an optical examination device according to a first embodiment. FIGS. 2A and 2B are views showing a positional relationship between an optical fiber structure and a tube when the optical fiber structure of the optical examination device according to the first embodiment is inserted into the tube, which is a detection object (examination object), where FIG. 2A is an enlarged longitudinal cross-sectional view of a main portion of the optical fiber structure, and FIG. 2B is a cross-sectional view taken along line I-I′ in FIG. 2A, showing optical paths of irradiation light emitted from a first light emitting portion and reflected light from the tube received by a first light receiving portion.

As shown in FIG. 1, an optical examination device 1 includes: a light source 23; at least one light-emitting optical fiber 2 including a first light guide portion 22 that propagates light emitted from the light source 23 and a first light emitting portion 21 that irradiates a detection object 6 with the light propagated through the first light guide portion 22, as irradiation light Lt; at least one light-receiving optical fiber 3 including a first light receiving portion 31 that receives reflected light Lt′ obtained when the irradiation light Lt emitted from the first light emitting portion 21 is reflected by the detection object 6 and a second light guide portion 32 that propagates the reflected light Lt′ received by the first light receiving portion 31; and a light detecting portion 33 that detects the reflected light Lt′ that has propagated through the second light guide portion 32. Here, the optical examination device 1 may further include a display portion 34 that displays a detection value of the intensity of the reflected light Lt′ detected by the light detecting portion 33. The display portion 34 may be configured not only to display the detection value of the intensity of the reflected light Lt′ but also to perform a predetermined process on the reflected light Lt′ and display results of the process.

In addition, as shown in FIGS. 2A and 2B, the light-emitting optical fiber 2 and the light-receiving optical fiber 3 form an optical fiber structure 10 in which the first light emitting portion 21 and the first light receiving portion 31 are offset from each other in the fiber axial-direction X. At this time, the first light emitting portion 21 and the first light receiving portion 31 are fixedly coupled in a parallel arrangement.

Thus, as shown in FIG. 2A, the first light emitting portion 21 of the light-emitting optical fiber 2 and the first light receiving portion 31 of the light-receiving optical fiber 3 are arranged in parallel. Thus, the light-emitting optical fiber 2 and the light-receiving optical fiber 3 are configured by separate optical fibers, respectively, and thus noise due to Fresnel reflected light can be prevented. Furthermore, it can be expected by reduction and prevention of the noise that an SN ratio is improved when the reflected light Lt′ reflected from the detection object 6 is received.

Here, in particular, a tip of the first light receiving portion 31 is located closer to the first light guide portion 22 than a tip 2a of the first light emitting portion 21 or the tip of the first light emitting portion 21 is located closer to a second light guide portion 32 than the tip 3a of the first light receiving portion 31, whereby when the detection object 6 is irradiated with the irradiation light Lt, the irradiation light Lt emitted from the first light emitting portion 21 can be effectively prevented from being directly taken in by the first light receiving portion 31, and the reflected light Lt′ obtained by being reflected once or a plurality of times on the detection object 6 is taken in by the first light receiving portion 31 as shown in FIG. 2B, so that noise due to the intake of the irradiation light Lt can be reduced in the detection value of the intensity of the reflected light Lt′ taken in by the first light receiving portion 31.

### Configuration of Optical Fiber Structure

The optical fiber structure 10 includes the light-emitting optical fiber 2 and the light-receiving optical fiber 3. Here, the optical fiber structure 10 shown in FIG. 1 includes one light-emitting optical fiber 2 and one light-receiving optical fiber 3. Thus, it can be expected to prevent noise in the reflected light Lt′ due to Fresnel reflected light.

### Light-Emitting Optical Fiber

The light-emitting optical fiber 2 includes the first light guide portion 22 that propagates the light emitted from the light source 23, and the first light emitting portion 21 that irradiates the detection object 6 with the light propagated through the first light guide portion 22 as the irradiation light Lt. This makes it possible to propagate the light emitted from the light source 23 through the first light guide portion 22 with small loss. Furthermore, since the light emitted from the light source 23 is propagated with small loss, it can be expected that an inner surface of a tube serving as the detection object 6 is irradiated with more light, as the irradiation light Lt, from the first light emitting portion 21.

Here, the light-emitting optical fiber 2 is configured to propagate the light emitted from the light source 23 at least in the first light guide portion 22. An example of the light-emitting optical fiber 2 is an optical fiber having a fiber shape with a core-clad structure in which, at least in the first light guide portion 22, when viewed in a cross section perpendicular to a fiber axial-direction X, a core is located in a center and cladding covers a whole circumference of the core. At this time, the first light guide portion 22 of the light-emitting optical fiber 2 is preferably configured by an optical fiber of which cladding is not removed by scraping. When the first light guide portion 22 is configured by the optical fiber whose cladding is not removed by scraping, it can be expected that the light from the light source 23 propagates with small loss. On the other hand, the first light emitting portion 21 of the light-emitting optical fiber 2 can be shaped such that part or all of the cladding is removed by scraping. When the first light emitting portion 21 is configured by an optical fiber of which part or all of the cladding is removed by scraping, it can be expected that the light from the light source 23 emitted from the cladding-removed part is emitted as the irradiation light Lt. The light-emitting optical fiber 2 may be a shape of an optical waveguide formed on a substrate or the like, without being limited to a fiber shape.

From the viewpoint of shortening the optical path to the detection object 6 and reducing the loss of the reflected light Lt′, it is preferable that the first light emitting portion 21 of the light-emitting optical fiber 2 is configured so that the irradiation light Lt is emitted approximately perpendicular to the fiber axial-direction X of the light-emitting optical fiber 2. From such a viewpoint, it is preferable that the irradiation light Lt emitted from the first light emitting portion 21 is emitted from the side surface of the first light emitting portion 21.

In the first light emitting portion 21 of the light-emitting optical fiber 2, at least a part that emits the irradiation light Lt may be subjected to at least one selected from the following treatment (a) to (d):
(a) surface roughening treatment using hydrofluoric acid or organic solvents;
(b) patterning treatment using a laser;
(c) coating treatment of coating a cladding-removed part with a transparent resin containing a light diffusion substance such as titanium oxide; and
(d) fusion splicing of a coreless fiber or a light diffusion fiber to a tip.

A fusion-spliced portion due to (d) fusion splicing of a coreless fiber or a light diffusion fiber to a tip out of these kinds of treatment is preferably subjected to coating treatment with a transparent resin having a high refractive index, and when the light-emitting optical fiber 2 is made of a quartz fiber, the transparent resin used in the coating treatment at this time preferably has a refractive index higher than that of the quartz glass that constitutes the cladding 27 of the quartz fiber.

Furthermore, a portion of the light-emitting optical fiber 2 subjected to (a) surface roughening treatment or (b) patterning treatment may be further coated with a transparent resin from the viewpoint of ensuring mechanical strength. A refractive index of the transparent resin used at this time is not particularly limited.

The material of the light-emitting optical fiber 2 is not particularly limited, but it is preferably made of a quartz fiber or a plastic fiber, particularly from the viewpoint of facilitating insertion into a tubular body or the like that is the detection object 6. Out of these materials, since the quartz fiber has tension, when the quartz fiber is used for the light-emitting optical fiber 2, the light-emitting optical fiber 2 can be deep inserted into an inner space of the tubular body even when the tubular body serving as the detection object 6 has a small diameter. In addition, since the plastic fiber is flexible, when the plastic fiber is used for the light-emitting optical fiber 2, the light-emitting optical fiber 2 can be inserted into a part with a high curvature even when the tubular body serving as the detection object 6 has a part with a high curvature, such as a blood vessel.

An outer diameter of the light-emitting optical fiber 2 is preferably set depending on the detection object 6 of the optical examination device 1. For example, when the detection object 6 is an inner wall surface of a blood vessel, the outer diameter of the light-emitting optical fiber 2 is preferably 250 μm or less from the viewpoint of facilitating installation in a small-diameter tube in a balloon catheter that is inserted into the blood vessel.

### Light-Receiving Optical Fiber

The light-receiving optical fiber 3 includes the first light receiving portion 31 that receives the reflected light Lt′ obtained when the irradiation light Lt emitted from the first light emitting portion 21 is reflected by the detection object 6, and the second light guide portion 32 that propagates the reflected light Lt′ received by the first light receiving portion 31. Thus, the reflected light Lt′ from an inner surface of the tube serving as the detection object 6 is received by the first light receiving portion 31, and is propagated through the second light guide portion 32 with small loss, whereby it can be expected that the reflected light Lt′ is reflected in the examination result through the light detecting portion 33, which will be described below.

Here, the light-receiving optical fiber 3 is configured to propagate the reflected light Lt′ at least in the second light guide portion 32. An example of the light-receiving optical fiber 3 is an optical fiber having a fiber shape with a core-clad structure similar to the light-emitting optical fiber 2 described above. At this time, the second light guide portion 32 of the light-receiving optical fiber 3 is preferably configured by an optical fiber of which cladding is not removed by scraping. When the second light guide portion 32 is configured by the optical fiber whose cladding is not removed by scraping, it can be expected that the reflected light Lt′ is propagated with small loss. On the other hand, the first light receiving portion 31 of the light-receiving optical fiber 3 can be shaped such that part or all of the cladding is removed by scraping. When the first light receiving portion 31 is configured by an optical fiber of which part or all of the cladding is removed by scraping, it can be expected that the reflected light Lt′ is received from the part of the cladding which is removed by scraping.

The first light receiving portion 31 of the light-receiving optical fiber 3 may be subjected to surface roughening treatment using hydrofluoric acid, an organic solvent, or the like, at least on the surface of the part that receives the reflected light Lt′. The light-receiving optical fiber 3 may be a shape of an optical waveguide formed on a substrate or the like, without being limited to a fiber shape.

In the first light receiving portion 31 of the light-receiving optical fiber 3, at least a part that receives the reflected light Lt′ may be subjected to at least one selected from the following treatment (a) to (d):
(a) surface roughening treatment using hydrofluoric acid or organic solvents;
(b) patterning treatment using a laser;
(c) coating treatment of coating a cladding-removed part with a transparent resin containing a light diffusion substance such as titanium oxide; and
(d) fusion splicing of a coreless fiber or a light diffusion fiber to a tip.

A fusion-spliced portion due to (d) fusion splicing of a coreless fiber or a light diffusion fiber to a tip out of these kinds of treatment is preferably subjected to coating treatment with a transparent resin having a high refractive index, and when the light-receiving optical fiber 3 is made of a quartz fiber, the transparent resin used in the coating treatment at this time preferably has a refractive index higher than that of the quartz glass that constitutes the cladding 37 of the quartz fiber.

Furthermore, a portion of the light-receiving optical fiber 3 subjected to (a) surface roughening treatment or (b) patterning treatment may be further coated with a transparent resin from the viewpoint of ensuring mechanical strength. A refractive index of the transparent resin used at this time is not particularly limited.

The material of the light-receiving optical fiber 3 is not particularly limited, but it is preferably made of a quartz fiber or a plastic fiber, particularly from the viewpoint of facilitating insertion into a tubular body or the like that is the detection object 6. Out of these materials, since the quartz fiber has tension, when the quartz fiber is used for the light-receiving optical fiber 3, the light-receiving optical fiber 3 can be deep inserted into an inner space of the tubular body even when the tubular body serving as the detection object 6 has a small diameter. In addition, since the plastic fiber is flexible, when the plastic fiber is used for the light-receiving optical fiber 3, the light-receiving optical fiber 3 can be inserted into a part with a high curvature even when the tubular body serving as the detection object 6 has a part with a high curvature, such as a blood vessel. Here, when the above-described light-emitting optical fiber 2 is made of a quartz fiber, the light-receiving optical fiber 3 is preferably made of a plastic fiber. Furthermore, when the above-described light-emitting optical fiber 2 is made of a plastic fiber, the light-receiving optical fiber 3 is preferably made of a quartz fiber. Since the light-emitting optical fiber 2 and the light-receiving optical fiber 3 are made of different materials, respectively, as described above and thus the quartz fiber with high tension and the plastic fiber with high flexibility can be combined, it is possible to adjust the tension and flexibility when a lateral force is applied to the optical fiber structure 10. Furthermore, by adjustment of the tension and flexibility when the lateral force is applied to the optical fiber structure 10, it can be expected that the optical fiber structure 10 is easily installed in a small-diameter tube in a balloon catheter that is inserted into the blood vessel. On the other hand, from the viewpoint of increasing tension of the optical fiber structure 10 when the light-emitting optical fiber 2 and the light-receiving optical fiber 3 are inserted into a catheter or the like, both of the light-emitting optical fiber 2 and the light-receiving optical fiber 3 may be made of a quartz optical fiber. Furthermore, from the viewpoint of increasing flexibility of the optical fiber structure 10 when the light-emitting optical fiber 2 and the light-receiving optical fiber 3 are inserted into a catheter or the like, both of the light-emitting optical fiber 2 and the light-receiving optical fiber 3 may be made of a plastic fiber.

In the optical examination device 1 shown in FIG. 1, a length dimension P₂ of the first light receiving portion 31 of the light-receiving optical fiber 3 is configured to be equal to a length dimension P₁ of the first light emitting portion 21 of the light-emitting optical fiber 2. Particularly, in the optical examination device 1 in which the optical fiber structure 10 is inserted into the blood vessel, both of the length dimension P₁ of the first light emitting portion 21 and the length dimension P₂ of the first light receiving portion 31 may be the same length in a range of 5 mm or more and 50 mm or less, and may be 30 mm as an example. On the other hand, for example, when the irradiation light It is emitted from the first light emitting portion 21 at a wide angle, the length dimension P₁ of the first light emitting portion 21 may be 5 mm or less.

An outer diameter of the light-receiving optical fiber 3 is preferably set depending on the detection object 6 of the optical examination device 1, similarly to the light-emitting optical fiber 2. For example, the outer diameter of the light-receiving optical fiber 3 is preferably 250 μm or less from the viewpoint of facilitating installation into a small-diameter tube. The optical inspection device 1 shown in FIG. 1 is configured so that the outer diameter of the light-receiving optical fiber 3 is equal to the outer diameter of the light-emitting optical fiber 2, but these outer diameters may be different from each other.

The light-receiving optical fiber 3 to be used preferably has a high numerical aperture, from the viewpoint of propagating the reflected light Lt′ with which the first light receiving portion 31 is irradiated from a wider range of directions to the light detecting portion 33 and increasing the amount of light received by the light detecting portion 33. As an example, the light-receiving optical fiber 3 may have a higher numerical aperture than that of the light-emitting optical fiber 2.

The light-emitting optical fiber 2 and the light-receiving optical fiber 3 form the optical fiber structure 10 in such a manner that the first light emitting portion 21 and the first light receiving portion 31 are offset from each other in the fiber axial-direction X. For example, in the optical inspection device 1 shown in FIG. 1, the first light emitting portion 21 and the first light receiving portion 31 are arranged to be entirely offset from each other in the fiber axial-direction X, thereby forming the optical fiber structure 10. In this way, the first light emitting portion 21 and the first light receiving portion 31 are arranged to be partially or entirely offset from each other to form the optical fiber structure 10, and thus the first light emitting portion 21 and the first light receiving portion 31 are fixedly coupled in a parallel arrangement, whereby the irradiation light Lt emitted from the first light emitting portion 21 is effectively prevented from being directly taken in by the first light receiving portion 31, and it can be expected that the optical fiber structure 10 can be easily installed in a tube or inserted into the detection object 6 without changing the positional relationship between the first light emitting portion 21 and the first light receiving portion 31.

### Arrangement of Light-Emitting Optical Fiber and Light-Receiving Optical Fiber

In the optical inspection device 1 shown in FIG. 1, the light-emitting optical fiber 2 and the light-receiving optical fiber 3 are preferably configured so that the tip of the first light receiving portion 31 is located closer to the first light guide portion 22 than the tip 2a of the first light emitting portion 21. More preferably, as shown in optical inspection device 1 shown in FIG. 2A, the tip of the first light receiving portion 31 of the light-receiving optical fiber 3 is located closer to the first light guide portion 22 of the light-emitting optical fiber 2 than the entire first light emitting portion 21. Thus, when the detection object 6 is irradiated with the irradiation light Lt, the irradiation light Lt emitted from the first light emitting portion 21 is not directly taken in by the first light receiving portion 31, and the detection object 6 is irradiated with the irradiation light, whereby the irradiation light Lt emitted from the first light emitting portion 21 can be effectively prevented from being directly taken in by the first light receiving portion 31. At the same time, the irradiation light Lt with which the detection object 6 is irradiated is reflected once or a plurality of times by the detection object 6 as shown in FIGS. 2A and 2B, to become the reflected light Lt′, which is taken in by the first light receiving portion 31. As described above, the reflected light Lt′ from the detection object 6 is taken in by the first light receiving portion 31, while the irradiation light Lt from the first light emitting portion 21 is effectively prevented from being directly taken in, thereby reducing noise due to the intake of the irradiation light Lt in the detection value of the intensity of the reflected light Lt′. Furthermore, it can be expected by reduction in the noise that an SN ratio of the light detected by the light detecting portion 33 is improved.

The first light emitting portion 21 and the first light receiving portion 31 are preferably fixedly coupled to each other in such a manner that the light emission from the first light emitting portion 21 and the light reception by the first light receiving portion 31 are not hindered. Examples of a means for fixing and coupling these components may include not only boding with an adhesive, but also a means for fixing the vicinity of the first light emitting portion 21 and the first light receiving portion 31 with a tube such as a heat-shrinkable tube or a means for crimping and fixing the vicinity of the first light emitting portion 21 and the first light receiving portion 31 using a ring or the like.

From the viewpoint of further facilitating installation of the optical fiber structure 10 in the tube or insertion of the optical fiber structure into the detection object 6, a fixing portion 5 may be provided that fixedly couples the light-emitting optical fiber 2 and the light-receiving optical fiber 3.

### Configuration of Optical Inspection Device

As shown in FIG. 1, the optical inspection device 1 includes at least the light source 23, the light-emitting optical fiber 2, the light-receiving optical fiber 3, and the light detecting portion 33, and the light-emitting optical fiber 2 and the light-receiving optical fiber 3 form the optical fiber structure 10 in which the first light emitting portion 21 and the first light receiving portion 31 are arranged to be offset from each other in the fiber axial-direction X. In other words, the optical inspection device 1 includes at least the light source 23 and the light detecting portion 33 in addition to the configuration of the optical fiber structure 10 described above.

Out of these components, the light source 23 is a light source of the irradiation light Lt. The light emitted from the light source 23 may be light containing a single wavelength component, such as laser light, or may be light containing a plurality of wavelength components.

The wavelength of the light emitted from the light source 23 is preferably selected from wavelengths that allow the light to propagate through the light-emitting optical fiber 2 and the light-receiving optical fiber 3, depending on the material of the detection object 6 and a medium between the first light emitting portion 21 and the detection object 6. For example, when the optical fiber structure 10 is used to block a blood flow in such a manner that a balloon catheter having the optical fiber structure arranged therein is inserted into the blood vessel and the balloon is inflated, the wavelength of the light emitted from the light source 23 is preferably a wavelength in a visible range (a wavelength of 400 nm or more and 700 nm or less) which is a wavelength allowing the light to be highly absorbed by blood.

In addition, the light emitted from the light source 23 may be a continuous wave to be emitted by CW oscillation, or may be a pulse wave to be emitted by pulse oscillation.

The light detecting portion 33 is a portion that detects the reflected light Lt′ propagating through the second light guide portion 32 of the light-receiving optical fiber 3. The light detecting portion 33 is configured by, for example, an optical sensor, and is configured to measure the intensity of the reflected light Lt′ and read out an electrical signal corresponding to the intensity. Here, the light detecting portion 33 may be provided in several numbers from the viewpoint of being capable of detecting the intensity of the reflected light Lt′ for each wavelength component, and in this case, the light detecting portion 33 may include a plurality of optical sensors corresponding to respective wavelength components.

Here, from the viewpoint of further increasing the SN ratio with respect to the reflected light, the light emitted from the light source 23 may be modulated (pulsed), and the reflected light Lt′ propagated through the second light guide portion 32 may be transmitted through an appropriate frequency filter to separate the reflected light Lt′ from the noise, and the reflected light Lt′ transmitted through the appropriate frequency filter may be detected by the light detecting portion 33.

The display portion 34 is a portion that displays the detection value of the intensity of the reflected light Lt′ detected by the light detecting portion 33. The display portion 34 is configured to display the detection value of the intensity of the received light to a person making measurement, based on the electrical signal from the light detecting portion 33. Here, when a plurality of light detecting portions 33 are provided, the display portion 34 may be provided in several numbers for the light detecting portions 33.

The optical examination device 1 may further include a light detecting portion and a display portion at positions branched from the light from the light source 23, on the light source side of the light-emitting optical fiber 2, the light detecting portion and the display portion not being shown. Thus, return light from the light-emitting optical fiber 2 is detected, whereby the amount of loss in the light-emitting optical fiber 2 and the light-receiving optical fiber 3 can be predicted. Furthermore, by the prediction of the amount of loss, it is also expected that examination accuracy of the optical examination device 1 will be improved.

### Uses of Optical Inspection Device and Optical Fiber Structure

The optical inspection device 1 and the optical fiber structure 10 can be widely used for inspection purposes by detecting the reflected light Lt′ from the detection object 6 when the detection object 6 is irradiated with the irradiation light Lt from the first light receiving portion 31.

As an example, when temporarily blocking blood flow in a blood vessel during surgery, the optical fiber structure 10 can be used to block the blood flow in such a manner that a balloon catheter having the optical fiber structure arranged therein is inserted into the blood vessel and the balloon is inflated. Thus, it is possible to detect the presence or absence of blood between a blood vessel wall and the balloon based on difference in reflectance of light between the blood vessel wall serving as the detection object and the blood. Furthermore, by the detection of the presence or absence of the blood between the blood vessel wall and the balloon, it can be expected that the blood flow in the blood vessel is appropriately blocked and damage to the blood vessel due to excessive inflation of the balloon hardly occurs. At this time, the optical fiber structure 10 may be used in a state of being inserted into a medical catheter or the like.

When the optical fiber structure 10 is arranged inside the balloon catheter, from the viewpoint of efficiently receiving the reflected light Lt′ from the blood vessel wall, it is desirable that one or both of the first light guide portion 22 of the light-emitting optical fiber 2 and the first light receiving portion 31 of the light-receiving optical fiber 3 are arranged in the balloon to be adjustable. Here, one or both of the first light guide portion 22 and the first light receiving portion 31 may be configured to be able to move in parallel in the fiber axial-direction X, and may also be configured to be rotatable around the central axis of the light-emitting optical fiber 2 or the light-receiving optical fiber 3 as a rotation axis. At this time, one or both of the first light guide portion 22 and the first light receiving portion 31 more preferably have a structure in which they can be fixed again after being adjusted in the arrangement in the balloon.

As another example, when temporarily stopping the flow of water in a pipe during water-supply works, the optical fiber structure 10 can be used to stop the flow of water in the pipe in such a manner that an airbag having the optical fiber structure arranged therein is inserted into the pipe and the airbag is inflated. Thus, it is possible to detect the presence or absence of water between a pipe wall and the airbag based on difference in reflectance of light depending on the presence or absence of water between the pipe wall serving as the detection object and the airbag. Furthermore, by the detection of the water between the pipe wall and the airbag, it can be expected that the flow of water in the pipe can be appropriately stopped and damage to the pipe or the airbag due to excessive inflation of the airbag hardly occurs.

### Second Embodiment

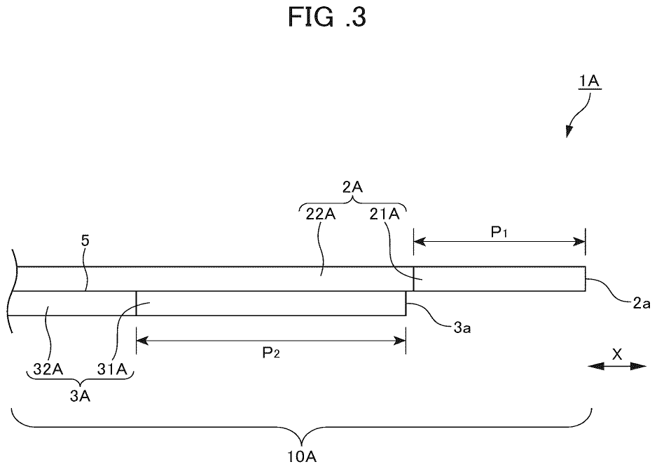

FIG. 3 is a plan view showing a structure of an optical fiber structure according to a second embodiment. Respective components shown in FIG. 3 are denoted by the same reference numerals when being the same as the components of the optical examination device 1 and the optical fiber structure 10 shown in FIG. 1.

The optical examination device 1 shown in FIG. 1 is configured in which the length dimension P₁ of the first light emitting portion 21 of the light-emitting optical fiber 2 is equal to the length dimension P₂ of the first light receiving portion 31 of the light-receiving optical fiber 3, but the present embodiment is not limited thereto. For example, as shown in an optical examination device 1A in FIG. 3, a length dimension P₂ of a first light receiving portion 31A of a light-receiving optical fiber 3A may be configured to be longer than a length dimension P₁ of a first light emitting portion 21A of a light-emitting optical fiber 2A. When the first light emitting portion 21A and the first light receiving portion 31A are configured in this way, the reflected light Lt′ can be received over a wider range of the light-receiving optical fiber 3A, thereby increasing the amount of light to be received by the light detecting portion 33.

Particularly, in the optical examination device 1A in which the optical fiber structure 10A is inserted into a blood vessel, the length dimension P₁ of the first light emitting portion 21A can be set to a range of 5 mm or more and 40 mm or less, and the length dimension P₂ of the first light receiving portion 31A can be set to a range of 10 mm or more and 50 mm or less. As an example, the length dimension P₁ of the first light emitting portion 21A can be set to 20 mm, and the length dimension P₂ of the first light receiving portion 31A can be set to 30 mm. Here, from the viewpoint of effectively increasing the reflected light Lt′ at the first light receiving portion 31A, the length dimension P₂ of the first light receiving portion 31A is preferably set to a range of 1.5 to 4.0 times the length dimension P₁ of the first light emitting portion 21A.

The optical inspection device 1 may be configured so that the length dimension P₂ of the first light receiving portion 31 of the light-receiving optical fiber 3 is shorter than the length dimension P₁ of the first light emitting portion 21 of the light-emitting optical fiber 2.

### Third Embodiment

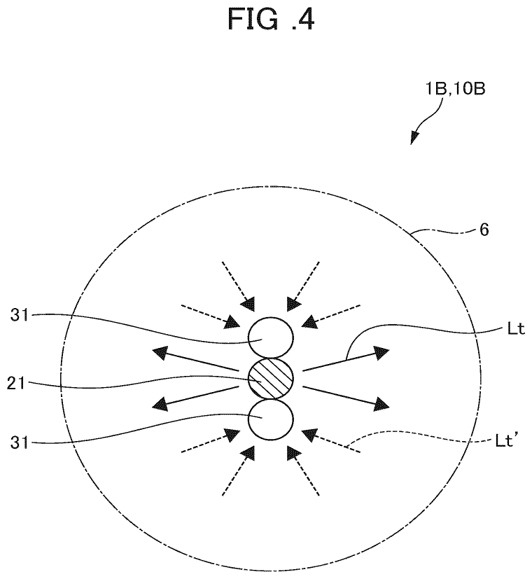

FIG. 4 is a cross-sectional view showing an arrangement of a light-emitting optical fiber and a light-receiving optical fiber that form an optical inspection device and an optical fiber structure according to a third embodiment. Respective components shown in FIG. 4 are denoted by the same reference numerals when being the same as the components of the optical inspection device 1 and the optical fiber structure 10 shown in FIG. 1.

The optical inspection device 1 shown in FIG. 1 is configured in which the optical fiber structure 10 includes one light-emitting optical fiber 2 and one light-receiving optical fiber 3, but the present embodiment may include, without being limited thereto, one or both of light-emitting optical fibers 2 and light-receiving optical fibers 3, which are provided in plurality. For example, as shown in an optical inspection device 1B in FIG. 4, an optical fiber structure 10B may include two or more light-receiving optical fibers 3 including first light receiving portions 31, respectively. Thus, the optical fiber structure 10B can receive a larger amount of reflected light Lt′ using a plurality of light-receiving optical fibers 3. As these plurality of light-receiving optical fibers 3 receive a larger amount of reflected light Lt′, it can be expected that the reflected light Lt′ propagated to the light detecting portion 33 is further increased.

Furthermore, the optical fiber structure 10 of the optical examination device 1 may include two or more light-receiving optical fibers 3 including the first light receiving portions 31, respectively. Thus, the detection object 6 is irradiated with the irradiation light Lt with a larger amount. As the detection object 6 is irradiated with the irradiation light It with a larger amount, it can be expected to obtain the intensity of the reflected light Lt′ capable of obtaining detection results with high accuracy in the light detecting portion 33, even when the reflectance of the irradiation light Lt on the detection object 6 is low in particular.

In addition, the optical examination device 1 may be configured in which the optical fiber structure 10 includes two or more light-emitting optical fibers 2 and two or more light-receiving optical fibers 3.

Here, one or more light-emitting optical fibers 2 and one or more light-receiving optical fibers 3 can form the optical fiber structure 10 in which the first light emitting portion 21 and the first light receiving portion 31 are arranged to be offset from each other in the fiber axial-direction X. For example, on one side of the light-emitting optical fiber 2 (upper side in FIG. 4) in the optical inspection device 1B in FIG. 4, the first light receiving portion 31 is arranged to be offset from and in parallel to the first light emitting portion 21 in the fiber axial-direction X, and on the other side of the light-emitting optical fiber 2 (lower side in FIG. 4), the first light receiving portion 31 is also arranged to be offset from and in parallel to the first light emitting portion 21 in the fiber axial-direction X, whereby the optical fiber structure 10B can be formed.

### Fourth Embodiment

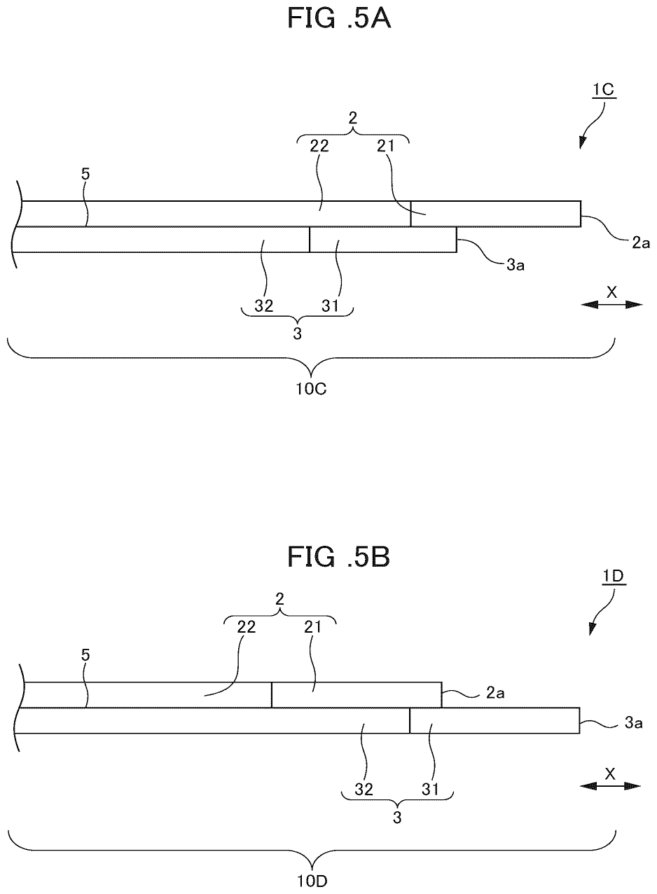

FIGS. 5A and 5B are plan views showing a structure of an optical fiber structure according to a fourth embodiment, where FIG. 5A shows a case where a tip of a first light receiving portion is arranged to be located closer to a first light guide portion than a tip of a first light emitting portion, and FIG. 5B shows a case where the tip of the first light emitting portion is arranged to be located closer to a second light guide portion than the tip of the first light receiving portion. Respective components shown in FIGS. 5A and 5B are denoted by the same reference numerals when being the same as the components of the optical inspection device 1 and the optical fiber structure 10 shown in FIG. 1.

In the optical inspection device 1 shown in FIG. 1, the light-emitting optical fiber 2 and the light-receiving optical fiber 3 form the optical fiber structure 10 in such a manner that the first light emitting portion 21 and the first light receiving portion 31 are arranged to be entirely offset from each other in the optical fiber axial-direction X, but the first light emitting portion 21 and the first light receiving portion 31 are arranged to be partially or entirely offset from each other in the optical fiber axial-direction X without being limited thereto, whereby an optical fiber structure 10 can be formed. For example, as shown in an optical inspection device 1C in FIG. 5A, the light-emitting optical fiber 2 and the light-receiving optical fiber 3 may form the optical fiber structure 10C in such a manner that the first light emitting portion 21 and the first light receiving portion 31 are arranged to be partially offset from each other in the fiber axial-direction X. At this time, in the optical fiber structure 10C, the first light emitting portion 21 and the first light receiving portion 31 are arranged to partially overlap with each other in the fiber axial-direction X.

Here, as shown in the optical inspection device 1C in FIG. 5A, the optical fiber structure 10C may be configured in which a tip 3a of the first light receiving portion 31 is arranged to be located closer to a first light guide portion 22 than a tip 2a of the first light emitting portion 21. On the other hand, as shown in the optical inspection device 1D in FIG. 5B, the optical fiber structure 10D may be configured in which a tip 2a of the first light emitting portion 21 is arranged to be located closer to a second light guide portion 32 than a tip 3a of the first light receiving portion 31.

### Fifth Embodiment

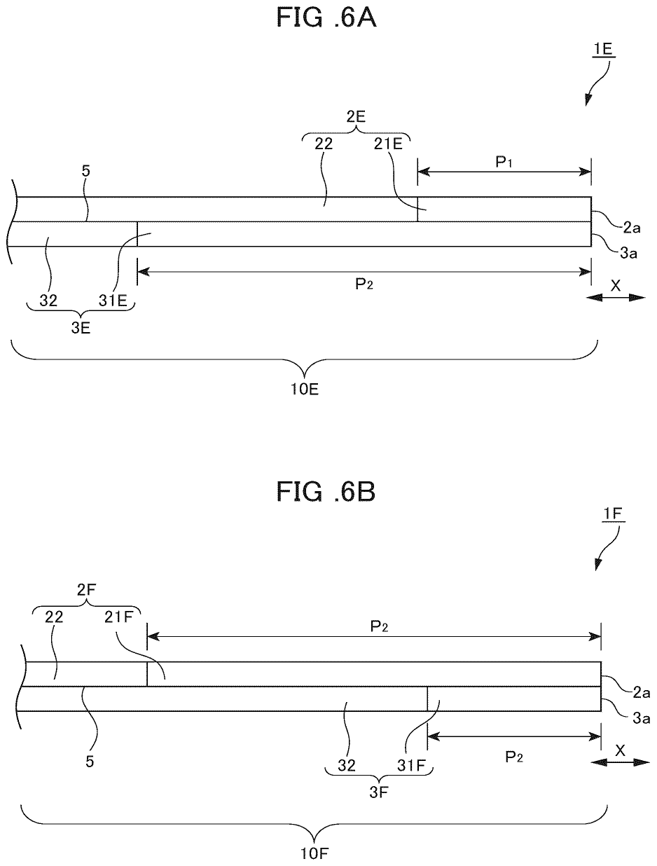

FIGS. 6A and 6B are plan views showing a structure of an optical fiber structure according to a fifth embodiment, where FIG. 6A shows a case where a length dimension of a first light receiving portion of a light-receiving optical fiber is longer than a length dimension of a first light emitting portion of a light-emitting optical fiber, and FIG. 6B shows a case where a length dimension of a first light emitting portion of a light-emitting optical fiber is longer than a length dimension of a first light receiving portion of a light-receiving optical fiber. Respective components shown in FIGS. 6A and 6B are denoted by the same reference numerals when being the same as the components of the optical inspection device 1 and the optical fiber structure 10 shown in FIG. 1.

In the optical inspection device 1 shown in FIGS. 5A and 5B described above, regarding the configuration in which the first light emitting portion 21 and the first light receiving portion 31 are partially offset from each other in the fiber axial-direction X, the length dimension P₁ of the first light emitting portion 21 of the light-emitting optical fiber 2 is configured to be equal to the length dimension P₂ of the first light receiving portion 31 of the first light-receiving optical fiber 3, but the length dimension P₁ of the first light emitting portion 21 of the light-emitting optical fiber 2 may be different from the length dimension P₂ of the first light receiving portion 31 of the first light-receiving optical fiber 3 without being limited thereto. For example, as shown in an optical inspection device 1E in FIG. 6A, regarding the configuration in which a first light emitting portion 21E and a first light receiving portion 31E are partially offset from each other in the fiber axial-direction X, a length dimension P₂ of the first light receiving portion 31E of a light-receiving optical fiber 3E may be configured to be longer than a length dimension P₁ of the first light emitting portion 21E of a first light-emitting optical fiber 2E.

At this time, as shown in the optical inspection device 1E in FIG. 6A, an optical fiber structure 10E may be configured in which the light-emitting optical fiber 2E and the light-receiving optical fiber 3E are arranged so that a tip 2a of the first light emitting portion 21E and a tip 3a of the first light receiving portion 31E are located at the same position in the fiber axial-direction X.

On the other hand, as shown in the optical inspection device 1F in FIG. 6B, an optical fiber structure 10F may be configured so that a first light emitting portion 21F and a first light receiving portion 31F partially overlap with each other in the fiber axial-direction X and a length dimension P₂ of the first light receiving portion 31F of a light-receiving optical fiber 3F is shorter than a length dimension P₁ of the first light emitting portion 21F of a light-emitting optical fiber 2F.

### Sixth Embodiment

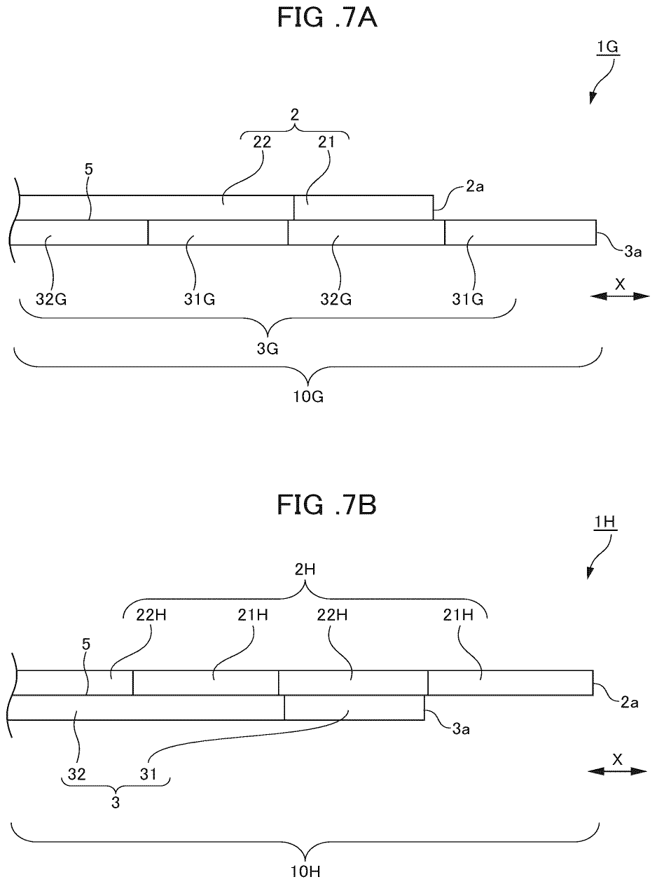

FIGS. 7A and 7B are plan views showing a structure of an optical fiber structure according to a sixth embodiment, where FIG. 7A shows a case where a light-receiving optical fiber includes a plurality of first light receiving portions, and FIG. 7B shows a case where a light-emitting optical fiber includes a plurality of first light emitting portions. Respective components shown in FIGS. 7A and 7B are denoted by the same reference numerals when being the same as the components of the optical inspection device 1 and the optical fiber structure 10 shown in FIG. 1.

In the optical inspection device 1 shown in FIG. 1, the light-emitting optical fiber 2 includes one first light emitting portion 21 and the light-receiving optical fiber 3 includes one first light receiving portion 31, but one or both of a first light emitting portion 21 and a first light receiving portion 31 may be provided in plurality. For example, as shown in an optical inspection device 1G in FIGS. 7A and 7B, a light-receiving optical fiber 3G may include a plurality of first light receiving portions 31G and 31G. At this time, a second light guide portion 32G is arranged between the plurality of first light receiving portions 31G and 31G. When the light-receiving optical fiber 3G includes the plurality of first light receiving portions 31G and 31G in this way, the reflected light Lt′ can be received over a wider range of the light-receiving optical fiber 3G, thereby increasing the amount of light to be received by the light detecting portion 33.

Here, the plurality of first light receiving portions 31G and 31G are preferably arranged so as to straddle a first light emitting portion 21G. Thus, even when the irradiation light Lt is emitted from the first light emitting portion 21G toward both sides in the fiber axial-direction X, the reflected light Lt′ can be received by the first light receiving portions 31G and 31G on both sides of the first light emitting portion 21G.

On the other hand, as shown in an optical inspection device 1H in FIG. 7B, an optical fiber structure 10H may be configured in which a light-emitting optical fiber 2H may include a plurality of first light emitting portions 21H and 21H. At this time, a first light guide portion 22H is arranged between the plurality of first light emitting portions 21H and 21H.

### Seventh Embodiment

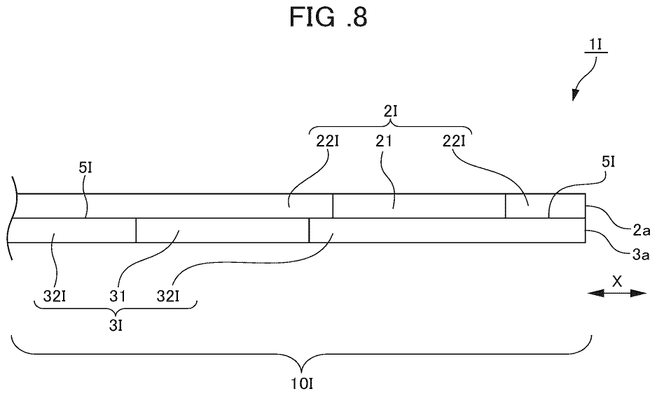

FIG. 8 is a plan view showing a structure of an optical fiber structure according to a seventh embodiment. Respective components shown in FIG. 8 are denoted by the same reference numerals when being the same as the components of the optical inspection device 1 and the optical fiber structure 10 shown in FIG. 1.

In the optical inspection device 1 shown in FIG. 1, the first light emitting portion 21 is provided at the end of the light-emitting optical fiber 2 and the first light receiving portion 31 is provided at the end of the light-receiving optical fiber 3, but one or both of a first light emitting portion 21 and a first light receiving portion 31 may be provided in an intermediate part of an optical fiber without being limited thereto. For example, as shown in an optical inspection device 1I in FIG. 8, an optical fiber structure 10I may be configured in which the first light emitting portion 21 is provided in an intermediate part of the light-emitting optical fiber 2I. In the optical fiber structure 10I, the first light receiving portion 31 may be provided in an intermediate part of a light-receiving optical fiber 3I. At this time, first light guide portions 22I and 22I are arranged on both sides of the first light emitting portion 21 in the fiber axial-direction X. In addition, second light guide portions 32I and 32I are arranged on both sides of the first light receiving portion 31 in the fiber axial-direction X.

Here, the optical fiber structure 10I preferably includes fixing portions 5I, which fixedly couple portions of the light-emitting optical fiber 2 and the light-receiving optical fiber 3, at ends of the light-emitting optical fiber 2I and the light-receiving optical fiber 3. Since the fixing portions 5I and 5I are provided at positions on both sides in the fiber axial-direction X from the first light receiving portion 31 and the first light emitting portion 21, the optical fiber structure 10I can be more easily installed in the tube or inserted into the detection object.

### Another Embodiment

In the above-described embodiments, the first light guide portion 22 of the light-emitting optical fiber 2 is configured by an optical fiber of which the cladding is not removed by scraping, and the first light emitting portion 21 of the light-emitting optical fiber 2 has a shape in which a part or all of the cladding of the optical fiber is removed by scraping, but the present embodiment is not limited thereto.

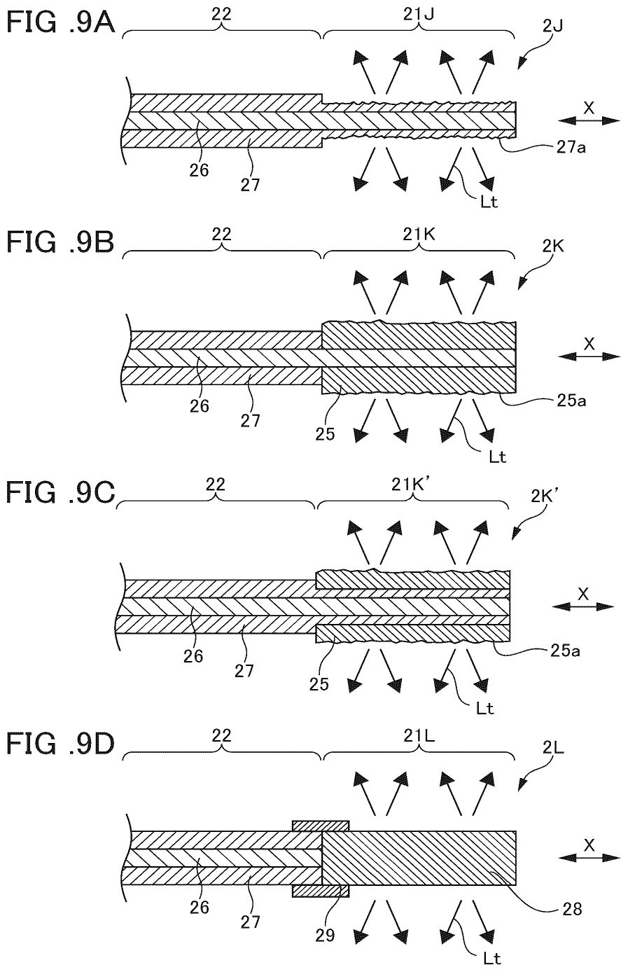

For example, as shown in a light-emitting optical fiber 2J in FIG. 9A, a first light emitting portion 21J of the light-emitting optical fiber 2J may have a shape in which a part of a cladding 27 of the optical fiber is removed by scraping, and a rough surface 27a may be formed on a surface of the cladding 27 which is removed by scraping. When the rough surface 27a is formed on the surface of the cladding 27 in this way, irradiation with the irradiation light Lt can be made from the first light emitting portion 21J in various directions, such as oblique directions relative to the fiber axial-direction X of the light-emitting optical fiber 2J. Here, the rough surface 27a can be formed on the surface of the cladding 27, which is removed by scraping, of the first light emitting portion 21J by surface roughening treatment using hydrofluoric acid or an organic solvent, or by patterning treatment using a laser. Furthermore, since the part of the light-emitting optical fiber 2J where the rough surface 27a is formed tends to have low mechanical strength due to small wire diameter, the part where the rough surface 27a is formed may be coated with a transparent resin (not shown) in order to increase the mechanical strength of the part. A refractive index of the transparent resin used in this case is not particularly limited.

As shown in a light-emitting optical fiber 2K in FIG. 9B and a light-emitting optical fiber 2K′ in FIG. 9C, a first light emitting portion 21K of the light-emitting optical fiber 2K (or a first light emitting portion 21K′ of the light-emitting optical fiber 2K′) is removed by scraping in whole or in part in a thickness direction of the cladding 27 of the optical fiber, that is, in a radial direction of the light-emitting optical fiber 2F (or the light-emitting optical fiber 2K′), a light-diffusion resin layer 25 having a light diffusing property is formed on the surface of a core 26 (or the cladding 27 which is removed by scraping), and a rough surface 25a may be further formed on a surface of the light-diffusion resin layer 25 as necessary. When the light-diffusion resin layer 25, which diffuses light, is formed on the surface of the core 26 or the cladding 27 in this way, irradiation with the irradiation light Lt can be made from the first light emitting portion 21K (or the first light emitting portion 21K′) in various directions, such as oblique directions relative to the fiber axial-direction X of the light-emitting optical fiber 2K (or the light-emitting optical fiber 2K′). Since the paths of the reflected light Lt′ reflected by the detection object 6 become more diverse due to the irradiation with the irradiation light Lt in various directions, it can be expected that the first light receiving portion 31 easily receives the reflected light Lt′. Here, the light-diffusion resin layer 25 formed on the surface of the core 26 or the cladding 27 is preferably made of a transparent resin containing a light-diffusion material such as titanium oxide, and the light-diffusion resin layer 25 can be formed by, for example, coating treatment of coating a part of the cladding 27 which is removed by scraping, with a transparent resin. Furthermore, the rough surface 25a can be formed on the surface of the light-diffusion resin layer 25 by surface roughening treatment using hydrofluoric acid or organic solvents, or by patterning treatment using a laser.

As shown in a light-emitting optical fiber 2L in FIG. 9D, a first light emitting portion 21L of the light-emitting optical fiber 2L may be formed by fusion splicing of a coreless fiber or a light diffusion fiber 28 to a tip of an optical fiber including a core 26 and a cladding 27. At this time, a part where the coreless fiber or the light diffusion fiber 28 is fusion-spliced tends to have low mechanical strength. Therefore, from the viewpoint of increasing the mechanical strength of the fusion-spliced part, at least the fusion-spliced part may be coated with a transparent or opaque resin layer 29. Here, the resin layer 29 coating the fusion-spliced part is preferably made of a transparent resin from the viewpoint of extracting more irradiation light Lt from the coreless fiber or the light diffusion fiber 28, and is more preferably made of a transparent resin having a high refractive index from the viewpoint of increasing the SN ratio of the irradiation light Lt to be extracted, in particular. Here, the transparent resin having a high refractive index preferably has a refractive index higher than that of quartz glass that constitutes the cladding 27 of the quartz fiber, for example, when the optical fiber to which the coreless fiber or the light diffusion fiber 28 is fusion-spliced is made of a quartz fiber. In addition, when the resin layer 29 is made of a transparent resin, the transparent resin may contain a light diffusion material such as titanium oxide. Furthermore, the resin layer 29 is preferably made of a material suitable for medical use, taking the possibility of balloon bursting into consideration.

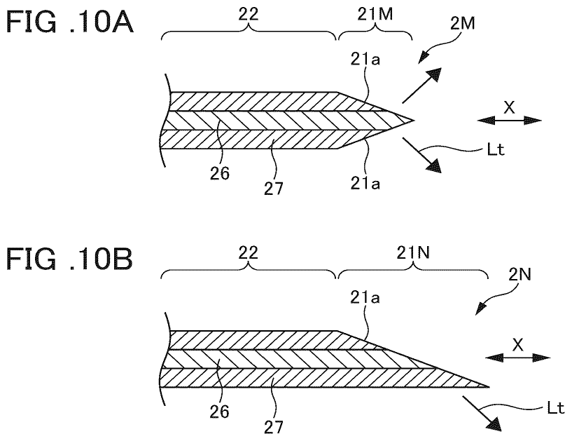

In particular, when a reflector or the like, which changes the traveling direction of the irradiation light Lt to a direction opposite to the fiber axial-direction X, is provided ahead of the tip 2a of the first light emitting portion 21 of the light-emitting optical fiber 2, for example, as shown in a light-emitting optical fiber 2M in FIG. 10A, a first light emitting portion 21M of the light-emitting optical fiber 2M may be configured by an inclined surface 21a obtained by cutting the core 26 and the cladding 27 of the optical fiber. Furthermore, for example, as shown in a light-emitting optical fiber 2N in FIG. 10B, a first light emitting portion 21N of the light-emitting optical fiber 2N may be configured by an inclined surface 21a obtained by cutting the core 26 and the cladding 27 of the optical fiber into a conical shape concentric with the core 26.

On the other hand, in the above-described embodiments, the second light guide portion 32 of the light-receiving optical fiber 3 is configured by an optical fiber of which the cladding is not removed by scraping, and the first light receiving portion 31 of the light-receiving optical fiber 3 has a shape in which a part or all of the cladding of the optical fiber is removed by scraping, but the present embodiment is not limited thereto.

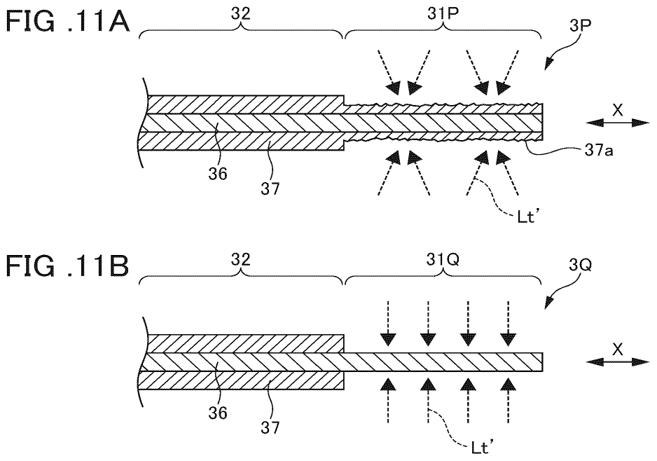

For example, as shown in a light-receiving optical fiber 3P in FIG. 11A, a first light receiving portion 31P of the light-receiving optical fiber 3P may have a shape in which a part of a cladding 37 of the optical fiber is removed by scraping, and a rough surface 34a may be formed on a surface of the cladding 37 which is removed by scraping. When the rough surface 34a is formed on the surface of the cladding 37 in this way, the reflected light Lt′ can be received by the first light receiving portion 31P in various directions, such as oblique directions relative to the fiber axial-direction X of the light-receiving optical fiber 3P. Since the first light receiving portion 31 receives the reflected light Lt′ in various direction, it can be expected to increase the possibility that first light receiving portion 31P can receive the reflected light Lt′.

As shown in a light-receiving optical fiber 3Q in FIG. 11B, a first light receiving portion 31Q of the light-receiving optical fiber 3Q can be shaped such that a part or all of the cladding 37 of the optical fiber is removed by scraping.

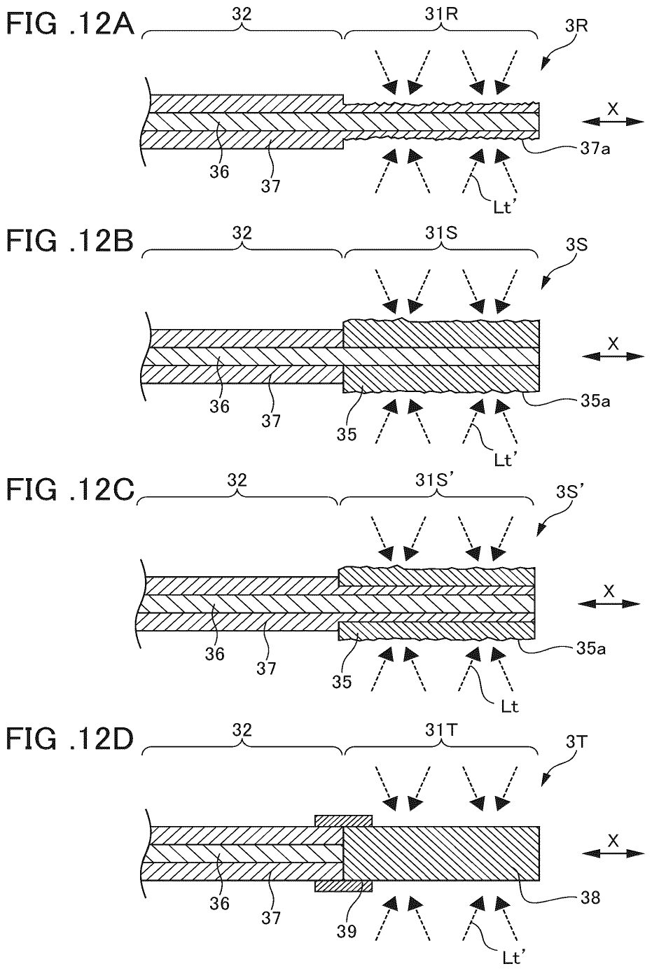

For example, as shown in a light-receiving optical fiber 3R in FIG. 12A, a first light receiving portion 31R of the light-receiving optical fiber 3R may have a shape in which a part of a cladding 37 of the optical fiber is removed by scraping, and a rough surface 37a may be formed on a surface of the cladding 37 which is removed by scraping. When the rough surface 37a is formed on the surface of the cladding 37 in this way, it is possible to receive reflected light Lt′ with which the first light receiving portion 31R is irradiated in various directions, such as oblique directions relative to the fiber axial-direction X of the light-receiving optical fiber 3R. Here, the rough surface 37a can be formed on the surface of the cladding 37, which is removed by scraping, of the first light receiving portion 31R by surface roughening treatment using hydrofluoric acid or an organic solvent, or by patterning treatment using a laser. Furthermore, since the part of the light-receiving optical fiber 3R where the rough surface 37a is formed tends to have low mechanical strength due to small wire diameter, the part where the rough surface 37a is formed may be coated with a transparent resin (not shown) in order to increase the mechanical strength of the part. A refractive index of the transparent resin used in this case is not particularly limited.

As shown in a light-receiving optical fiber 3S in FIG. 12B and a light-receiving optical fiber 3S′ in FIG. 12C, a first light receiving portion 31S of the light-receiving optical fiber 3S (or a first light receiving portion 31S′ of the light-receiving optical fiber 3S′) is removed by scraping in whole or in part in a thickness direction of the cladding 37 of the optical fiber, that is, in a radial direction of the light-receiving optical fiber 3S (or the light-receiving optical fiber 3S′), a light-diffusion resin layer 35 having a light diffusing property is formed on the surface of a core 36 (or the cladding 37 which is removed by scraping), and a rough surface 35a may be further formed on a surface of the light-diffusion resin layer 35 as necessary. When the light-diffusion resin layer 35, which diffuses light, is formed on the surface of the core 36 or the cladding 37 in this way, irradiation with the irradiation light Lt can be made from the first light receiving portion 31S (or the first light receiving portion 31S′) in various directions, such as oblique directions relative to the fiber axial-direction X of the light-receiving optical fiber 3S (or the light-receiving optical fiber 3S′). Since the paths of the reflected light Lt′ reflected by the detection object 6 become more diverse due to the irradiation with the irradiation light Lt in various directions, it can be expected that the first light receiving portion 31 easily receives the reflected light Lt′. Here, the light-diffusion resin layer 35 formed on the surface of the core 36 or the cladding 37 is preferably made of a transparent resin containing a light-diffusion material such as titanium oxide, and the light-diffusion resin layer 35 can be formed by, for example, coating treatment of coating the core 36 or a part of the cladding 37 which is removed by scraping, with a transparent resin. Furthermore, the rough surface 35a can be formed on the surface of the light-diffusion resin layer 35 by surface roughening treatment using hydrofluoric acid or organic solvents, or by patterning treatment using a laser.

As shown in a light-receiving optical fiber 3T in FIG. 12D, a first light receiving portion 31T of the light-receiving optical fiber 3T may be formed by fusion splicing of a coreless fiber or a light diffusion fiber 38 to a tip of an optical fiber including a core 36 and a cladding 37. At this time, a part where the coreless fiber or the light diffusion fiber 38 is fusion-spliced tends to have low mechanical strength. Therefore, from the viewpoint of increasing the mechanical strength of the fusion-spliced part, at least the fusion-spliced part may be coated with a transparent or opaque resin layer 39. Here, the resin layer 39 coating the fusion-spliced part is preferably made of a transparent resin from the viewpoint of allowing a larger amount of reflected light Lt′ to be incident on the coreless fiber or the light diffusion fiber 38, and is more preferably made of a transparent resin having a high refractive index from the viewpoint of increasing the SN ratio of the reflected light Lt′ to be incident, in particular. Here, the transparent resin having a high refractive index preferably has a refractive index higher than that of quartz glass that constitutes the cladding 37 of the quartz fiber, for example, when the optical fiber to which the coreless fiber or the light diffusion fiber 38 is fusion-spliced is made of a quartz fiber. In addition, when the resin layer 39 is made of a transparent resin, the transparent resin may contain a light diffusion material such as titanium oxide. Furthermore, the resin layer 39 is preferably made of a material suitable for medical use, taking the possibility of balloon bursting into consideration.

Although embodiments of the present invention have been described above, the present invention is not limited to the above-described embodiments, but includes all aspects included in the concept of the present invention and the scope of the claims, and can be modified in various ways within the scope of the present invention.

### EXPLANATION OF REFERENCE NUMERALS

1, 1A to 1I optical inspection device
10, 10A to 10I optical fiber structure
2, 2A, 2E to 2F, 2H to 2K, 2K′, 2L to 2N light-emitting optical fiber
2a tip of light-emitting optical fiber
21, 21A, 21E to 21F, 21H, 21J, 21K, 21K′, 21L to 21N first light emitting portion
21a inclined surface
22, 22A, 22H, 22I first light guide portion
23 light source
25, 35 light-diffusion resin layer
25a, 35a rough surface of light-diffusion resin layer
26 core of light-emitting optical fiber
27 cladding of light-emitting optical fiber
27a rough surface of cladding of light-emitting optical fiber
28, 38 coreless fiber or light diffusion fiber
29, 39 (transparent or opaque) resin layer
3, 3E to 3G, 3I, 3P to 3S, 3S′, 3T light-receiving optical fiber
3a tip of light-receiving optical fiber
31, 31A, 31E to 31G, 31P to 31S, 31S′, 31T first light receiving portion
32, 32A, 32G, 32I second light guide portion
33 light detecting portion
34 display portion
36 core of light-receiving optical fiber
37 cladding of light-receiving optical fiber
37a rough surface of cladding of light-emitting optical fiber
5 fixing portion
6 detection object
Lt irradiation light
Lt′ reflected light
P₁ length dimension of first light emitting portion
P₂ length dimension of first light receiving portion
X fiber axial-direction

1. An optical inspection device comprising:
a light source;
at least one light-emitting optical fiber including a first light guide portion that propagates light emitted from the light source and a first light emitting portion that irradiates a detection object with the light propagated through the first light guide portion, as irradiation light;
at least one light-receiving optical fiber including a first light receiving portion that receives reflected light obtained when the irradiation light emitted from the first light emitting portion is reflected by the detection object and a second light guide portion that propagates the reflected light received by the first light receiving portion; and
a light detecting portion that detects the reflected light propagated through the second light guide portion,
wherein
the light-emitting optical fiber and the light-receiving optical fiber form an optical fiber structure in which the first light emitting portion and the first light receiving portion are arranged to be offset from each other in a fiber axial-direction.

2. The optical inspection device according to claim 1, wherein the irradiation light is emitted from a side surface of the first light emitting portion.

3. The optical inspection device according to claim 1, wherein the first light receiving portion of the light-receiving optical fiber has a longer length than the first light emitting portion of the light-emitting optical fiber.

4. The optical inspection device according to claim 1, wherein the light-emitting optical fiber and the light-receiving optical fiber form the optical fiber structure in which the first light emitting portion and the first light receiving portion are arranged to be partially offset from each other in the fiber axial-direction.

5. The optical inspection device according to claim 1, wherein
the light-emitting optical fiber and the light-receiving optical fiber are respectively arranged so that a tip of the first light emitting portion and a tip of the first light receiving portion are at the same position in the fiber axial-direction.

6. The optical inspection device according to claim 1, wherein the light-receiving optical fiber includes a plurality of the first light receiving portions.

7. The optical inspection device according to claim 1, wherein the light-emitting optical fiber includes a plurality of the first light emitting portions.

8. The optical inspection device according to claim 1, wherein one or both of the first light emitting portion and the first light receiving portion are provided in an intermediate part of the light-emitting optical fiber or the light-receiving optical fiber.

9. The optical inspection device according to claim 1, wherein the light-emitting optical fiber and the light-receiving optical fiber are made of a quartz fiber or a plastic fiber.

10. An optical fiber structure comprising:
at least one light-emitting optical fiber including a first light guide portion that propagates light emitted from a light source and a first light emitting portion that irradiates a detection object with the light propagated through the first light guide portion, as irradiation light; and
at least one light-receiving optical fiber including a first light receiving portion that receives reflected light obtained when the irradiation light emitted from the first light emitting portion is reflected by the detection object and a second light guide portion that propagates the reflected light received by the first light receiving portion,
wherein
the light-emitting optical fiber and the light-receiving optical fiber form an optical fiber structure in which the first light emitting portion and the first light receiving portion are arranged to be offset from each other in a fiber axial-direction.

11. The optical inspection device according to claim 2, wherein the light-emitting optical fiber and the light-receiving optical fiber are made of a quartz fiber or a plastic fiber.

12. The optical inspection device according to claim 3, wherein the light-emitting optical fiber and the light-receiving optical fiber are made of a quartz fiber or a plastic fiber.

13. The optical inspection device according to claim 4, wherein the light-emitting optical fiber and the light-receiving optical fiber are made of a quartz fiber or a plastic fiber.

14. The optical inspection device according to claim 5, wherein the light-emitting optical fiber and the light-receiving optical fiber are made of a quartz fiber or a plastic fiber.

15. The optical inspection device according to claim 6, wherein the light-emitting optical fiber and the light-receiving optical fiber are made of a quartz fiber or a plastic fiber.

16. The optical inspection device according to claim 7, wherein the light-emitting optical fiber and the light-receiving optical fiber are made of a quartz fiber or a plastic fiber.

17. The optical inspection device according to claim 8, wherein the light-emitting optical fiber and the light-receiving optical fiber are made of a quartz fiber or a plastic fiber.
